(19) United States
(12) Patent Application Publication
(10) Pub. No.: US 2013/0165737 A1
(43) Pub. Date: Jun. 27, 2013

(54) IMPLANTATION OF A HEARING PROSTHESIS

(71) Applicant: Koen Van den Heuvel, Hove (BE)

(72) Inventor: Koen Van den Heuvel, Hove (BE)

(21) Appl. No.: 13/336,466

(22) Filed: Dec. 23, 2011

(51) Int. Cl. A61F 11/04; A61B 19/00; H04R 25/00; A61B 17/3209

(52) U.S. Cl. 600/25; 606/180; 606/130

(57) ABSTRACT

A method of implanting a hearing prosthesis in a recipient, including boring an artificial passageway into the temporal bone to a middle ear cavity based on a virtual model of at least a portion of a recipient's temporal bone.

### BACKGROUND

1. Field of the Invention

The present invention relates generally to hearing prostheses, and more particularly, to implantation of a hearing prosthesis.

2. Related Art

Hearing loss is generally of two types, conductive and sensorineural. Sensorineural hearing loss is due to the absence or destruction of the cochlear hair cells which transduce sound into nerve impulses. Various hearing prostheses have been developed to provide individuals suffering from sensorineural hearing loss with the ability to perceive sound. For example, cochlear implants have an electrode assembly which is implanted in the cochlea. In operation, electrical stimuli are delivered to the auditory nerve via the electrode assembly, thereby bypassing the inoperative hair cells to cause a hearing percept.

Conductive hearing loss occurs when the natural mechanical pathways that provide sound in the form of mechanical energy to cochlea are impeded, for example, by damage to the ossicular chain or ear canal. For a variety of reasons, such individuals are typically not candidates for a cochlear implant. Rather, individuals suffering from conductive hearing loss typically receive an acoustic hearing aid. Hearing aids rely on principles of air conduction to transmit acoustic signals to the cochlea. In particular, hearing aids amplify received sound and transmit the amplified sound into the ear canal. This amplified sound reaches the cochlea in the form of mechanical energy, causing motion of the perilymph and stimulation of the auditory nerve.

Unfortunately, not all individuals suffering from conductive hearing loss are able to derive suitable benefit from hearing aids. For example, some individuals are prone to chronic inflammation or infection of the ear canal. Other individuals have malformed or absent outer ear and/or ear canals resulting from a birth defect, or as a result of medical conditions such as Treacher Collins syndrome or Microtia.

For these and other individuals, another type of hearing prosthesis has been developed in recent years. This hearing prosthesis, commonly referred to as a middle ear implant, converts received sound into a mechanical force that is applied to the ossicular chain or directly to the cochlea via an actuator implanted in or adjacent to the middle ear cavity.

### SUMMARY

Some aspects of the present invention are generally directed to a method of implanting a hearing prosthesis in a middle ear cavity of a recipient, comprising boring an artificial passageway through the temporal bone to provide access to the middle ear cavity, wherein the boring is based on a virtual model of at least a portion of a recipient's temporal bone.

Some other aspects of the present invention are generally directed to a hearing prosthesis, comprising an implantable actuator support having a first surface configured to osseointegrate to a mastoid bone of a recipient and a second surface configured to removably receive a middle ear implant actuator.

Some other aspects of the present invention are generally directed to a hearing prosthesis, comprising an implantable component having an outer surface, wherein at least a portion of the outer surface is configured to abut bone of a recipient, the outer surface including a first sub-portion configured to osseointegrate and a second sub-portion configured to, relative to the first sub-portion, resist osseointegration.

### BRIEF DESCRIPTION OF THE DRAWINGS

Embodiments of the present invention are described below with reference to the attached drawings, in which:

FIG. 1 is perspective view of a human ear;

FIG. 2A is a perspective view of an exemplary direct acoustic cochlear stimulator implanted in accordance with embodiments of the present invention;

FIG. 2B is a perspective view of an exemplary direct acoustic cochlear stimulator implanted in accordance with an embodiment of the present invention;

FIG. 2C is a perspective view of an exemplary direct acoustic cochlear stimulator implanted in accordance with an embodiment of the present invention;

FIG. 3 is a schematic depicting an exemplary implantable component in accordance with an embodiment of the present invention;

FIG. 4A is a perspective view of an exemplary system according to embodiments of the present invention;

FIG. 4B is a simplified block diagram of an exemplary remote control unit for controlling the system of FIG. 4A, according to an embodiments of the present invention;

FIG. 4C is a cross-sectional view of an exemplary insertion tool for implanting an internal component of a middle ear implant, in accordance with embodiments of the present invention;

FIGS. 5A-5C are flowcharts according to exemplary embodiments of the present invention;

FIGS. 6A-6B depict an exemplary implantable actuator support according to exemplary embodiments of the present invention;

FIGS. 7A-7B depict another exemplary implantable actuator support according an exemplary embodiments of the present invention;

FIG. 8 depicts another exemplary implantable actuator support according to an exemplary embodiment of the present invention; and

FIGS. 9A-9B depict another exemplary implantable actuator support according an exemplary embodiments of the present invention.

### DETAILED DESCRIPTION

Aspects of the present invention are generally directed to implanting a component of a hearing prosthesis in a middle ear cavity of a recipient. The method includes developing a virtual model of the temporal bone adjacent the middle ear cavity; using the virtual model to bore an artificial passageway through the temporal bone to the middle ear cavity; and inserting the hearing prosthesis component through the passageway and into the middle ear cavity. At least a portion of the method may be executed in an automated fashion.

Aspects of the present invention are also generally directed to an implantable actuator support configured to be implanted in the passageway to support the actuator. As will be detailed below, the actuator support is configured to facilitate the replacement of the actuator at a time post implantation. In contrast, the implantable actuator support may stay in place for a substantially longer time period, including, for example, the post implantation lifespan of the recipient.

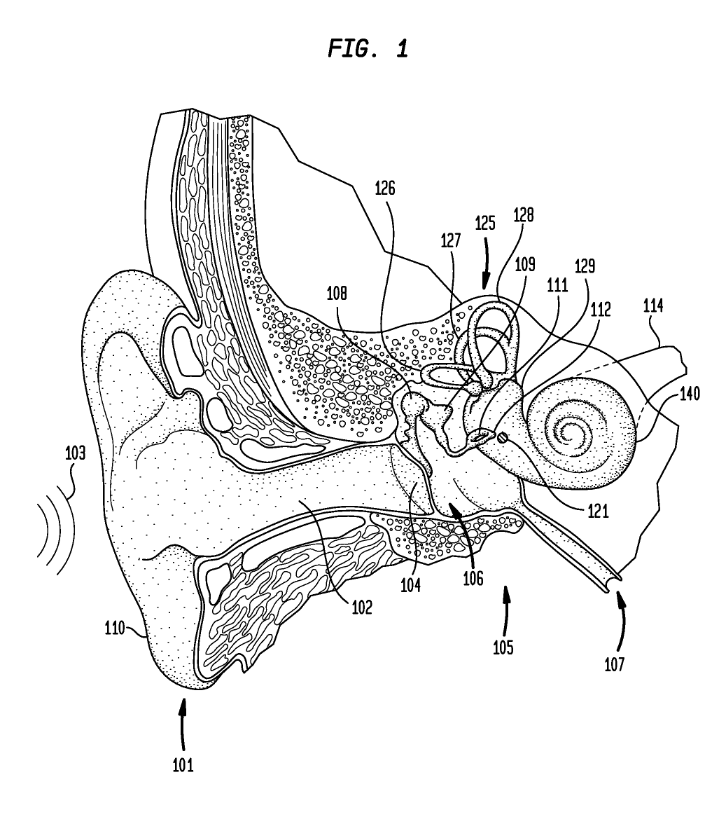

FIG. 1 is perspective view of a human skull showing the anatomy of the human ear. As shown in FIG. 1, the human ear comprises an outer ear 101, a middle ear 105 and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112, which is adjacent round window 121. This vibration is coupled through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to the vibration of tympanic membrane 104. This vibration sets up waves of fluid motion of the perilymph within cochlea 140. Such fluid motion, in turn, activates hair cells (not shown) inside cochlea 140. Activation of the hair cells causes nerve impulses to be generated and transferred through the spiral ganglion cells (not shown) and auditory nerve 114 to the brain (also not shown) where they cause a hearing percept.

As shown in FIG. 1, semicircular canals 125 are three half-circular, interconnected tubes located adjacent cochlea 140. Vestibule 129 provides fluid communication between semicircular canals 125 and cochlea 140. The three canals are the horizontal semicircular canal 126, the posterior semicircular canal 127, and the superior semicircular canal 128. The canals 126, 127 and 128 are aligned approximately orthogonally to one another. Specifically, horizontal canal 126 is aligned roughly horizontally in the head, while the superior 128 and posterior canals 127 are aligned roughly at a 45 degree angle to a vertical through the center of the individual's head.

Each canal is filled with a fluid called endolymph and contains a motion sensor with tiny hairs (not shown) whose ends are embedded in a gelatinous structure called the cupula (also not shown). As the orientation of the skull changes, the endolymph is forced into different sections of the canals. The hairs detect when the endolymph passes thereby, and a signal is then sent to the brain. Using these hair cells, horizontal canal 126 detects horizontal head movements, while the superior 128 and posterior 127 canals detect vertical head movements.

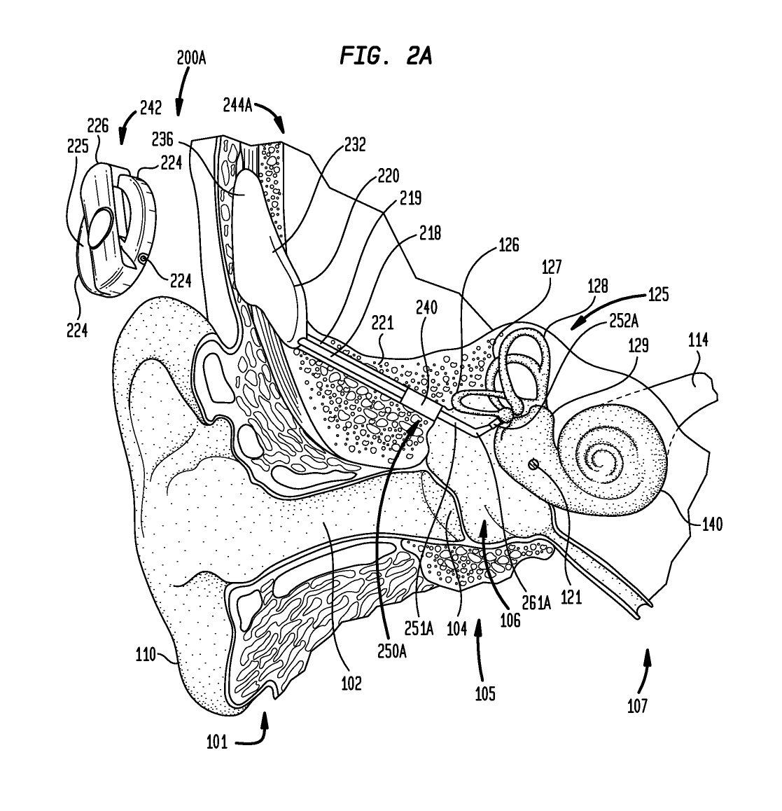

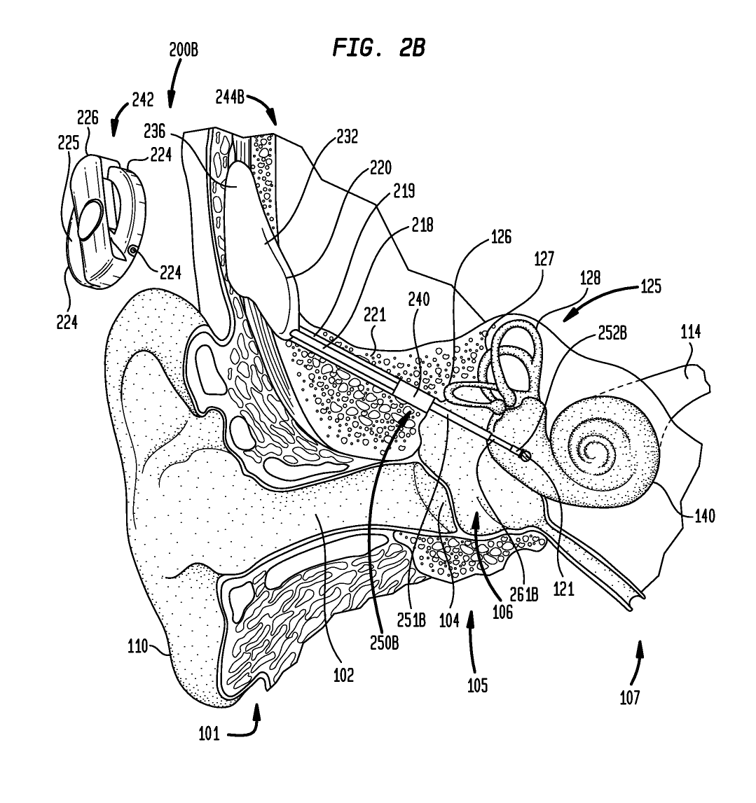

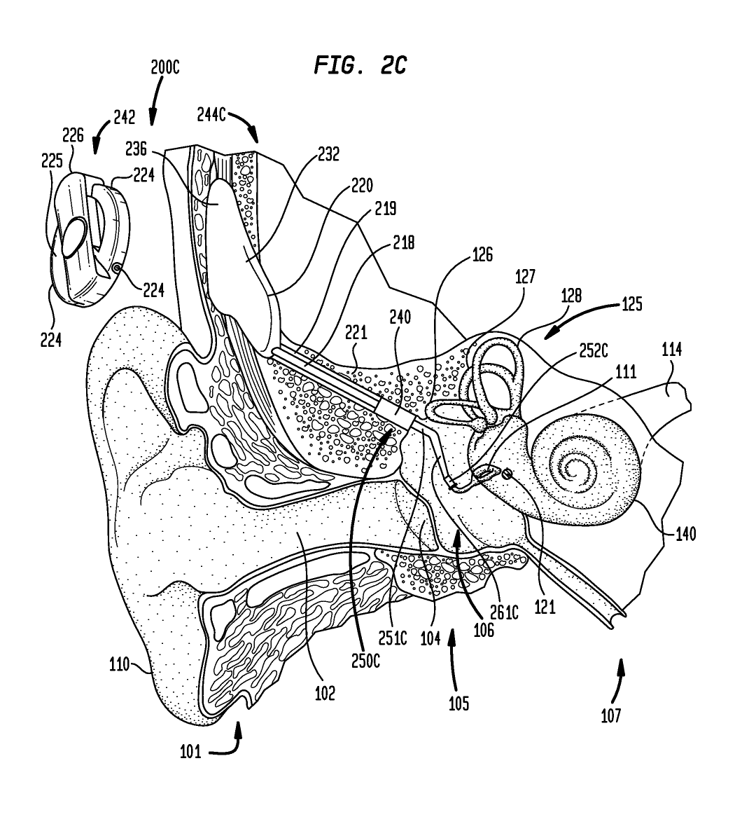

FIG. 2A is a perspective view of an exemplary direct acoustic cochlear stimulator 200A in accordance with embodiments of the present invention. Direct acoustic cochlear stimulator 200A comprises an external component 242 that is directly or indirectly attached to the body of the recipient, and an internal component 244A that is temporarily or permanently implanted in the recipient. External component 242 typically comprises two or more sound input elements, such as microphones 224, for detecting sound, a sound processing unit 226, a power source (not shown), and an external transmitter unit 225. External transmitter unit 225 comprises an external coil (not shown). Sound processing unit 226 processes the output of microphones 224 and generates encoded data signals which are provided to external transmitter unit 225. For ease of illustration, sound processing unit 226 is shown detached from the recipient.

Internal component 244A comprises an internal receiver unit 232, a stimulator unit 220, and a stimulation arrangement 250A in electrical communication with stimulator unit 220 via cable 218 extending thorough artificial passageway 219 in mastoid bone 221. Internal receiver unit 232 and stimulator unit 220 are hermetically sealed within a biocompatible housing, and are sometimes collectively referred to as a stimulator/receiver unit.

Internal receiver unit 232 comprises an internal coil (not shown), and optionally, a magnet (also not shown) fixed relative to the internal coil. The external coil transmits electrical signals (i.e., power and stimulation data) to the internal coil via a radio frequency (RF) link. The internal coil is typically a wire antenna coil comprised of multiple turns of electrically insulated platinum or gold wire. The electrical insulation of the internal coil is provided by a flexible silicone molding (not shown). In use, implantable receiver unit 232 is positioned in a recess of the temporal bone adjacent auricle 110.

In the illustrative embodiment of FIG. 2A, ossicles 106 have been explanted. However, it should be appreciated that stimulation arrangement 250A may be implanted without disturbing ossicles 106.

Stimulation arrangement 250A comprises an actuator 240, a stapes prosthesis 252A and a coupling element 251A which includes an artificial incus 261B. Actuator 240 is osseointegrated to mastoid bone 221, or more particularly, to the interior of artificial passageway 219 formed in mastoid bone 221.

In this embodiment, stimulation arrangement 250A is implanted and/or configured such that a portion of stapes prosthesis 252A abuts an opening in one of the semicircular canals 125. For example, in the illustrative embodiment, stapes prosthesis 252A abuts an opening in horizontal semicircular canal 126. In alternative embodiments, stimulation arrangement 250A is implanted such that stapes prosthesis 252A abuts an opening in posterior semicircular canal 127 or superior semicircular canal 128.

As noted above, a sound signal is received by microphone(s) 224, processed by sound processing unit 226, and transmitted as encoded data signals to internal receiver 232. Based on these received signals, stimulator unit 220 generates drive signals which cause actuation of actuator 240. The mechanical motion of actuator 240 is transferred to stapes prosthesis 252A such that a wave of fluid motion is generated in horizontal semicircular canal 126. Because, vestibule 129 provides fluid communication between the semicircular canals 125 and the median canal, the wave of fluid motion continues into median canal, thereby activating the hair cells of the organ of Corti. Activation of the hair cells causes appropriate nerve impulses to be generated and transferred through the spiral ganglion cells (not shown) and auditory nerve 114 to cause a hearing percept in the brain.

FIG. 2B is a perspective view of another type of direct acoustic cochlear stimulator 200B in accordance with an embodiment of the present invention. Direct acoustic cochlear stimulator 200B comprises external component 242 and an internal component 244B.

Stimulation arrangement 250B comprises actuator 240, a stapes prosthesis 252B and a coupling element 251 B which includes artificial incus 261 B which couples the actuator to the stapes prosthesis. In this embodiment, stimulation arrangement 250B is implanted and/or configured such that a portion of stapes prosthesis 252B abuts round window 121 of cochlea 140.

The embodiments of FIGS. 2A and 2B are exemplary embodiments of a middle ear implant that provides mechanical stimulation directly to cochlea 140. Other types of middle ear implants provide mechanical stimulation to middle ear 105. For example, middle ear implants may provide mechanical stimulation to a bone of ossicles 106, such to incus 109 or stapes 111. FIG. 2C depicts an exemplary embodiment of a middle ear implant 200C having a stimulation arrangement 250C comprising actuator 240 and a coupling element 251C. Coupling element 251C includes a stapes prosthesis 252C and an artificial incus 261C which couples the actuator to the stapes prosthesis. In this embodiment, stapes prosthesis 252C abuts stapes 111.

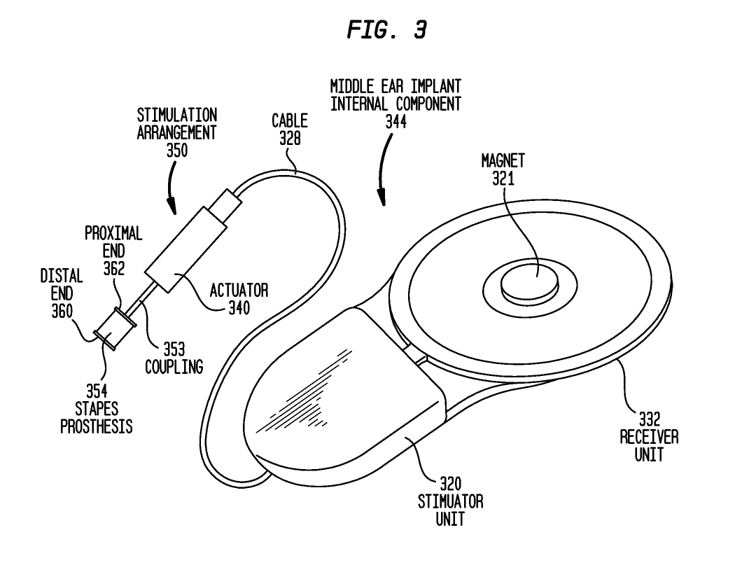

FIG. 3 is a perspective view of an exemplary internal component 344 of a middle ear implant which generally represents internal components 244 described above. Internal component 344 comprises an internal receiver unit 332, a stimulator unit 320, and a stimulation arrangement 350. As shown, receiver unit 332 comprises an internal coil (not shown), and preferably, a magnet 320 fixed relative to the internal coil. As would be appreciated, internal receiver unit 332 and stimulator unit 320 are typically hermetically sealed within a biocompatible housing. This housing has been omitted from FIG. 3 for ease of illustration.

Stimulator unit 320 is connected to stimulation arrangement 350 via a cable 328. Stimulation arrangement 350 comprises an actuator 340, a stapes prosthesis 354 and a coupling element 353. A distal end of stapes prosthesis 354 is configured to be positioned in one or more of the configurations noted above with respect to FIGS. 2A-2C. A proximal end of stapes prosthesis 354 is connected to actuator 340 via coupling 353 and the distal end of the prosthesis is directly or indirectly coupled to the cochlea. In operation, actuator 340 vibrates stapes prosthesis 354. The vibration of stapes prosthesis 354 generates waves of fluid motion of the perilymph, thereby activating the hair cells of the organ of Corti. Activation of the hair cells causes appropriate nerve impulses to be generated and transferred through the spiral ganglion cells and auditory nerve 114.

Aspects of the present invention are generally directed to implantation procedures for implanting components of a hearing prosthesis such as internal component 344 of a middle ear implant. Described next below is an exemplary boring system which may be utilized during such implantation procedures.

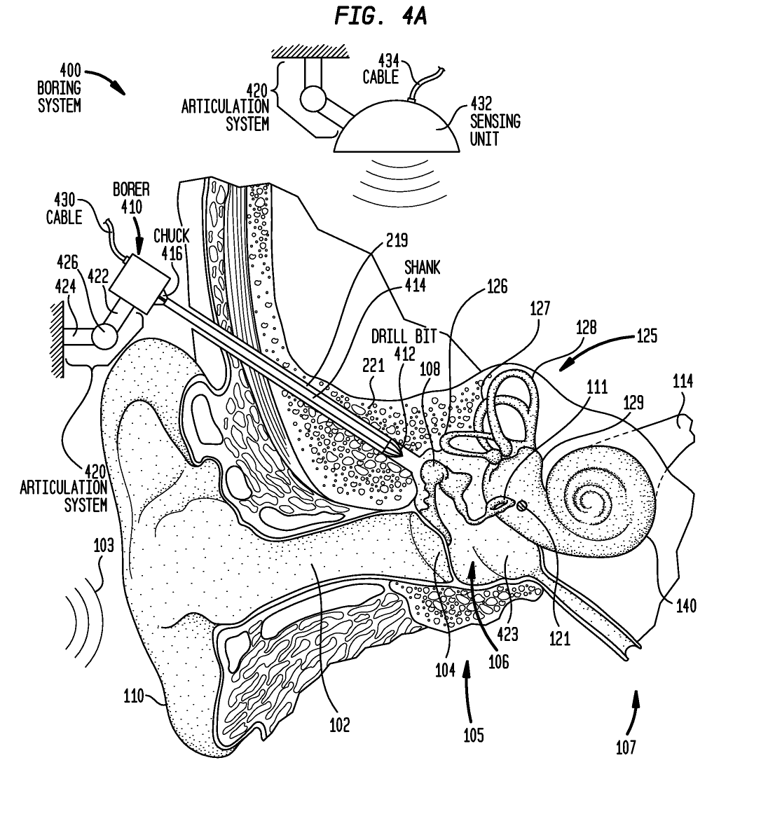

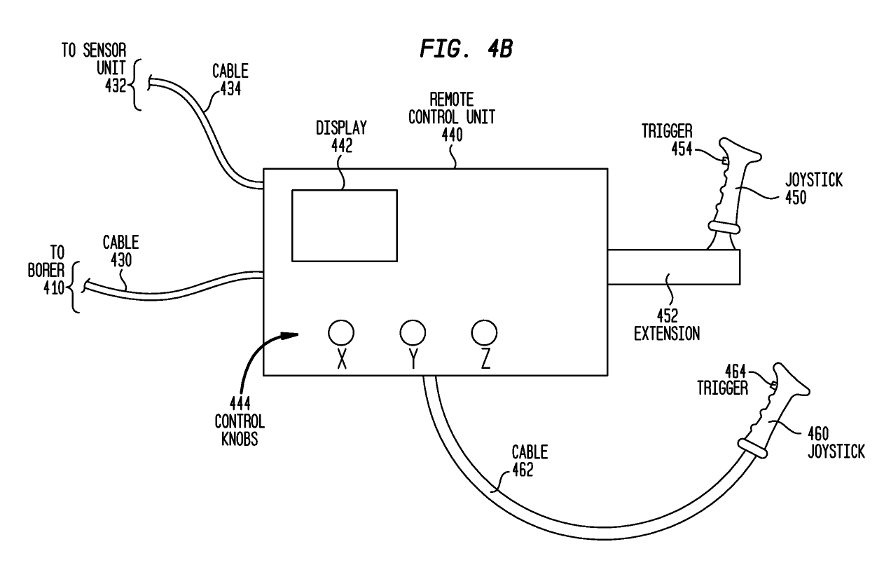

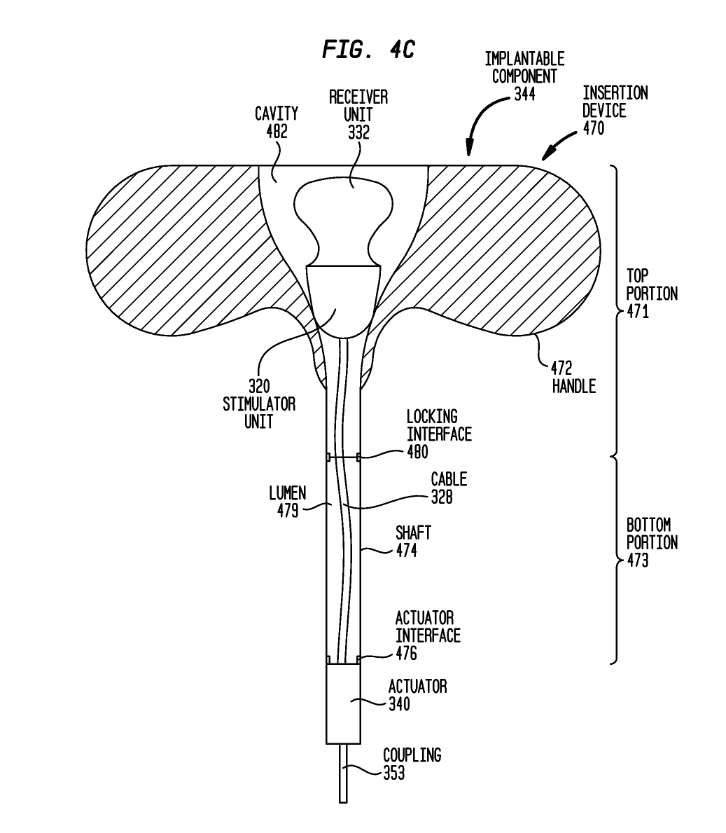

FIG. 4A is a perspective view of an exemplary embodiment of a boring system 400. System 400 includes a borer 410 to which drill bit 412 is connected via shank 414 which is releasably connected to chuck 416. Chuck 416 is connected to a drill motor (not shown) in borer 410. Borer 410 is mounted to support and movement system 420, comprising support arm 422 which is connected to joint 426 which in turn is connected to support arm 424. Support arm 424 is rigidly mounted to a wall, a floor, or some other relatively stationary surface. Joint 426 permits drill housing 418, and thus drill bit 412, to be moved in one, two, three, four, five or six degrees of freedom. In an exemplary embodiment, joint 426 includes actuators that move drill housing 418 in an automated manner, as will be described below. In an exemplary embodiment, cavity borer 410 is configured to drill from the outer surface of the mastoid bone 221 straight to the middle ear cavity 423. Cavity borer 410 is configured to be remotely controlled via communication with a remote control unit via communication lines of cable 430.

Artificial passageway establishment system further includes a sensor 432. While sensor 432 is depicted as being co-located with cavity borer 410, as detailed below, sensor 432 may be used relatively much prior to use of cavity borer 410. Sensing unit 432 is configured to scan the head of a recipient and obtain data indicative of spatial locations of internal organs (e.g., mastoid bone 221, middle ear cavity 423 and/or ossicles 106, etc.) In an exemplary embodiment, sensing unit 432 is a unit that is also configured to obtain data indicative of spatial locations of at least some components of the cavity borer 410, such as, for example, the drill bit 412. The obtained data may be communicated to remote control unit 440 via communication lines of cable 434. As may be seen, sensor 432 is mounted to a support and movement system 420 that may be similar to or the same as that used by the cavity borer 410.

In an exemplary embodiment, sensing unit 432 is an MRI system, an X-Ray system, an ultrasound system, or any other system which will permit the data indicative of the spatial locations to be determined as detailed herein and variations thereof. As will be described below, this data may be obtained prior to surgery and/or during surgery. It is noted that in some embodiments, at least some portions of the cavity borer 410 are configured to be better imaged or otherwise detected by sensing unit 432. In an exemplary embodiment, bit 412 includes radio-opaque contrast material. In an exemplary embodiment, at least some portions of cavity borer 410 are made of non-ferromagnetic material or other materials that are more compatible with an MRI system or another sensing unit utilized with the embodiment of FIG. 4A than ferromagnetic material or the like. As will be described in greater detail below, the data obtained by sensing unit 432 is used to construct a 3D or 4D model of the recipient's head and/or specific organs of the recipient's head (e.g., temporal bone) and/or portions of the cavity borer 410.

FIG. 4B is a simplified block diagram of an exemplary embodiment of a remote control unit 440 for controlling artificial passageway borer 410 and sensing unit 432 via communication lines 430 and 434, respectively. Remote control unit 440 includes a display 442 that displays a virtual image of the mastoid bone obtained from sensor 432 and may superimpose a virtual image of drill bit 412 onto the virtual image indicative of a current position of the drill bit relative to the ear anatomy. An operator (e.g., surgeon, certified healthcare provider, etc) utilizes remote control unit 440 to control some or all aspects of artificial passageway borer 412 and/or sensing unit 432. Exemplary control may include depth of drilling, angle of drilling, speed of advancement and/or retraction of drill bit 412, rotation speed of drill bit 412, rotation direction of drill bit 412, etc. Such control may be exercised via joystick 450 mounted on extension 452 which fixedly mounts joystick 450 to a control unit housing. Such control may be further exercised via joystick 460 which is not rigidly connected to housing of remote control unit 440. Instead, it is freely movable relative thereto and is in communication with the remote control unit via communication lines of cable 462. Joystick 462 may be part of a virtual system in which the remote control unit 440 extrapolates control commands based on how the joystick 462 is moved in space, or joystick may be a device that permits the operator more limited control over the cavity borer 410. Such control may include, for example an emergency stop upon release of trigger 464 and/or directing the cavity borer 410 to drill further into the mastoid bone by squeezing the trigger 464 (which, in some embodiments, may control a speed at which the drill bit 412 is advanced by squeezing harder and/or more on the trigger). In the same vein, trigger 454 of joystick 450 may have similar and or the same functionality.

Control of the cavity borer 410 may also be exercised via knobs 440 which may be used to adjust an angle of the drill bit 412 in the X, Y and Z axis, respectively. Other controls components may be included in remote control 440.

FIG. 4C is a cross-section of an exemplary insertion tool 470 configured to be used to implant and/or explant at least a portion of and/or all of an implantable component of a middle ear implant. In the exemplary embodiment depicted in FIG. 4C, insertion tool 470 includes a top portion 471 having handle 472 in the form of projections radially extending from a center axis 488 of the device. Insertion device 470 also includes a bottom portion 473 which may be removably attachable to top portion 471 via locking interface 480. Top portion 471 includes a cavity 482 located between handles 472 configured to receive receiver unit 332 and stimulator unit 320 of implantable component 344, as may be seen. Bottom portion 473 includes a cavity 474 configured to receive cable 328 therein. Bottom portion 476 includes actuator interface 476 which is configured to carry or otherwise apply a force to a rear side of actuator 340. Briefly, the insertion device 470 may be used to insert actuator 340 into artificial passageway 219 to a desired location therein and/or in the middle ear cavity 423, optionally while connected to receiver unit 332 and stimulator unit 320 via cable 328. In an exemplary embodiment, the top portion 471 may be released from the bottom portion 473 to facilitate insertion device 470 removal from the recipient while leaving behind actuator 340 and, optionally, the implantable component 344. In an exemplary embodiment, insertion device 470 includes a slit that runs the length of the device that permits the cable 328, the stimulator unit 320 and the receiver unit 332 to be removed therethrough (e.g., upon sufficient flexing of the insertion device 470 via, for example, a bending force applied to the insertion device 470 through handles 472 to widen the slit). In an exemplary embodiment, the insertion device 470 is configured such that a torque may be applied to the insertion device 470 through handles 470 and communicated to actuator 340, thus permitting actuator 340 to be screwed into position in artificial passageway 419. Such an insertion device 470 may have utility in embodiments where the actuator 340 includes threads.

It is noted that remote control unit 440, cavity borer 410, sensing unit 432 and insertion tool 470 are but exemplary embodiments of such devices. Any device, system and/or method that may be utilized to implement the surgical and implantation procedures detailed herein and/or variations thereof may be used in some embodiments. In this regard, an exemplary embodiment includes a method of utilizing some and/or all of the components detailed in FIGS. 4A, 4B and/or 4C to implant an implantable component of a hearing prosthesis. Along these lines, additional details of utilizing the artificial passageway establishment system will now be described.

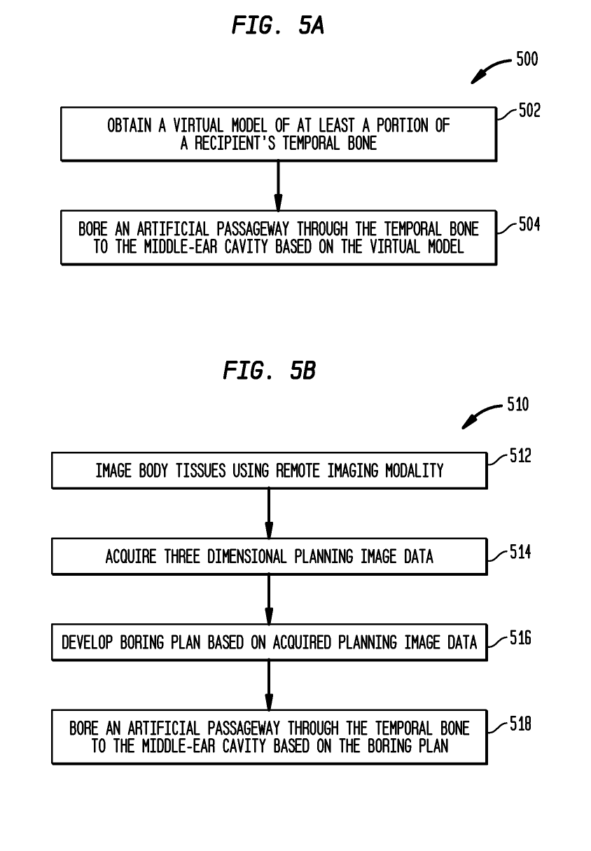

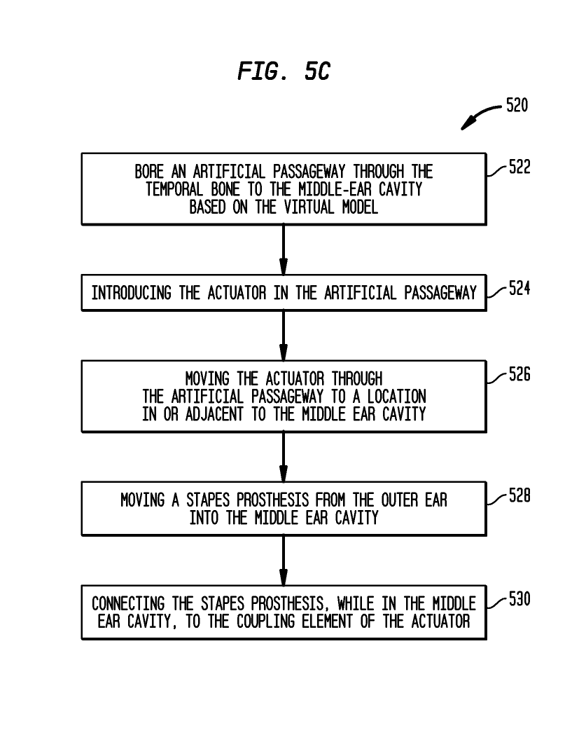

FIG. 5A depicts a high-level exemplary flowchart corresponding to an exemplary method 500 of implanting a hearing prosthesis in a recipient. At step 502, a virtual model of at least a portion of the recipient's temporal bone (e.g., mastoid bone) is obtained. This may be accomplished by utilizing sensing unit 432, which may be an MRI machine, and using data therefrom to construct the virtual model of the recipient's temporal bone and/or other body features related to the temporal bone (e.g., middle ear, inner ear, etc.) Such virtual models may be in the form of images from one or more MRI scans and/or a 3D model assembled from a plurality of MRI scans, in so-called “hard copy” print form and/or electronically displayed form. The virtual model may be a 4D model in that it may include movement features of the 3D model (e.g., due to the heartbeat cycle and/or the breathing cycle). The virtual model may be in the form of a dataset from which features such as geometric features and/or spatial features may be extrapolated. Any model of the temporal bone and/or other body features related to the temporal bone which will permit the methods detailed herein and variations thereof may be obtained to practice some embodiments.

Method 500 includes step 504, which entails boring artificial passageway 219 into the temporal bone to a middle ear cavity based on the virtual model. In an exemplary embodiment, step 504 includes guiding at least one of the direction of boring and a depth of boring relative to a surface of the temporal bone (e.g., the surface proximate the skin) utilizing an artificial guidance system established based on the virtual model. This artificial guidance system may be a mechanical guidance system or an electronic guidance system, with support and movement system 420 being an example of a combination mechanical guidance system and electronic guidance system. Along these lines, in embodiments of support and movement system 420 having actuators, support and movement system 420 corresponds to a robot, as will be described below.

FIG. 5B presents a more detailed method 510 according to an exemplary embodiment. At step 512, body tissues (e.g., the temporal bone) are imaged for planning purposes, typically using a remote imaging modality (e.g., sensing unit 432) such as a computed tomography (CT), magnetic resonance imaging (MRI), ultrasound imaging, X-ray imaging, PET, SPECT, optical coherence tomography, a combination of these, or other imaging modalities. At step 514, a three dimensional planning image data set (i.e., virtual model) encompassing the temporal bone is acquired from the imaging modality. It is noted that in some embodiments, all of the temporal bone and/or other body tissue proximate the temporal bone may no necessarily be visible in the image, so long as sufficiently contrasting surrogate structures are visible in the image data to permit boring of the artificial passageway 219 based on the image data set. In an exemplary embodiment, the location of some pertinent tissue structure not readily imaged can be sufficiently estimated with reference to structures that can be imaged and/or to an implanted surrogate/fiducial (discussed further below). The planning imaging used in many embodiments may include a time sequence of three-dimensional tissue volumes, with the time sequence typically spanning one or more movement cycles (such as a cardiac or heartbeat cycle, a respiration or breathing cycle, and/or the like). Accordingly, a three dimensional planning image data set may encompass a four dimensional planning image data set. In exemplary embodiments, the image data comprises a series of CT slices through the temporal bone, the middle ear and/or the inner ear so as to provide three-dimensional image data. The time series of three-dimensional images may be acquired at times that are distributed throughout the heartbeat cycle and/or respiratory cycle, so that the image planning data effectively comprises a time series of three-dimensional image datasets providing information regarding the motion of tissues during the respective cycles.

Referring still to FIG. 5B, from step 514, method 510 proceeds to step 516, which entails developing a boring plan based on the imaging data obtained from image step 514. The plan typically comprises movements of the cavity borer 410 that will result in the desired artificial passageway 219, and may include such features as depth of drilling, angle of drilling, speed of advancement and/or retraction of drill bit 412, rotation speed of drill bit 412, rotation direction of drill bit 412, etc. After the boring plan is developed, the method proceeds to step 518, where the artificial passageway 219 is bored based on the boring plan. It is noted that by boring the cavity based on the boring plan, this includes boring the cavity based on a virtual model of at least a portion of a recipient's temporal bone, as the boring plan is based on the virtual model.

It is noted that in some embodiments, actions 502, 512, 514 and 516 may be performed contemporaneously with actions 504 and 518. It is noted that in some embodiments, actions 502, 512, 514 and 516 may be performed prior to (e.g., hours, days, weeks, etc) to actions 504 and 518, and, as such, may be performed prior to obtaining access to the temporal bone through skin of the recipient (e.g., cutting into skin to reach bone). With regard to the former, an exemplary embodiment may sensing unit 432 being used in real-time with cavity borer 410, and the progress of cavity borer 412 may be monitored using sensing unit 432. In such an embodiment, implanted surrogates/fiducials may be used to aid in determining anatomical locations. By way of example, gold or platinum fiducials may be inserted through the outer ear into the middle ear cavity 423 and positioned therein at a desired location. The fiducials may be incorporated into the virtual model and thus may serve as, for example, a target for the boring operation. During boring, the location of the drill bit 412 may be imaged relative to the implanted fiducials to determine the accuracy of the boring operation.

The action of developing a boring plan may be performed prior to or contemporaneous with the action of boring. In this regard, an initial boring plan may be developed based on a previously obtained virtual model of tissue, and an updated boring plan may be developed in an iterative manner as the cavity borer 410 proceeds to bore into the temporal bone. In this regard, the updated boring plan (and the initial plan) may be obtained/developed automatically utilizing a computer, etc., or may be obtained/developed manually.

In an exemplary embodiment, an operator of the cavity borer 410 is furnished with a boring plan based on the virtual model and bores the artificial passageway 219 in accordance with the plan. In an alternative embodiment, cavity borer 410 automatically evaluates the virtual model and automatically develops a boring plan based on the virtual model. The boring plan is used by the cavity borer 410 in an automated process to bore the artificial passageway 219. In an alternative embodiment, the operator of the cavity borer 410 uses the plan to manually bore the artificial passageway 219.

As noted above, an exemplary method includes the action of guiding at least one of the direction of boring and a depth of boring relative to a surface of the temporal bone utilizing an artificial guidance system established based on the virtual model, where this artificial guidance system may be a mechanical and/or electronic guidance system. In an exemplary embodiment of a mechanical guidance system, a gantry or other support structure having two, three, four, five and/or six degrees of freedom may support cavity borer 410. The cavity borer 410 may be adjusted in space by adjusting the gantry to properly align the cavity borer 410 with the temporal bone and/or to drive the cavity borer 410 into and/or out of the temporal bone.

In an exemplary embodiment of an electronic guidance system, the cavity borer 410 may be fitted with LEDs or the like, and a remote sensing unit may be used to monitor the position and/or orientation of the cavity borer in space based on the location of the LEDs. Because a relationship between the LEDs and other pertinent portions of the cavity borer are known, information about the position and/or orientation of the cavity borer may be ascertained. Such an electronic alignment system may provide instructions to an operator to move the cavity borer 410 in a specified manner and/or may simply provide the operator with feedback as to the location of the cavity borer (e.g., superimposed on the virtual model of the temporal bone). Any device, system and/or method that will provide an artificial guidance system may be used in some embodiments herein and variations thereof.

In some embodiments, the methods herein and variations thereof may include programming an artificial guidance system based on the virtual model to automatically guide the cavity borer. In this regard, this may entail programming the guidance system based on the boring plan detailed above. Still further, an exemplary method may entail automatically boring the artificial passageway into the temporal bone utilizing the programmed guidance system. It is noted that in some embodiments, automatic guidance actions/automatic boring actions and manual guidance actions/manual boring actions are not mutually exclusive. For example, an exemplary embodiment may include manually guiding the cavity borer 410 while an automatic guidance system limits the ability of the operator to move the cavity borer 410 outside a predetermined envelope (e.g., the guidance system prevents the operator from driving drill bit 412 too deeply into the inner ear cavity 423.

FIG. 5C depicts an exemplary flowchart representing an exemplary method 520, which entails method step 522 corresponding to method step 514 above. Method 520 further includes step 524, which entails positioning actuator 340 in the artificial passageway 219, followed by method step 526, which entails moving the positioned actuator 340 through at least a portion of the artificial passageway to a location at least one of proximate or into the middle ear cavity (e.g., the position depicted in FIG. 2B). It is noted that in an exemplary embodiment, one or both of steps 524 and 526 may be accomplished with insertion device 470 detailed above with respect to FIG. 4C.

Method 520 further includes step 528, which entails moving a stapes prosthesis 252B from an outer ear 102 of the recipient into the middle ear cavity 423, the method step 528 being preceded by the action of establishing an opening through ear drum 104, if necessary. Following method step 528, method 520 includes the step of connecting the stapes prosthesis 252B, while in the middle ear cavity 423, to a coupling element 251A of the actuator 340

It is noted that in an exemplary embodiment, method step 530 may be preceded and/or executed simultaneously with the action of adjusting a location of the actuator 340 within the artificial passageway 219 and/or within the middle ear cavity 423. In an exemplary embodiment, this may be performed by applying a pushing force or a pulling force to insertion device 470, which is releasably attached to actuator 340, to respectively push and pull the actuator 340. In an exemplary embodiment, in the case of an actuator 340 having outer threads configured to thread into the bone wall of artificial passageway 219, twisting the insertion device 470 clockwise may drive the actuator 340 further towards the middle ear, and twisting the insertion device 470 counterclockwise may drive the actuator further away from the middle ear, or visa-versa, depending on the direction of threads of the actuator. Accordingly, an embodiment includes an actuator having a threaded portion configured to threadably interface with the bone wall of artificial passageway 219.

As noted, an exemplary embodiment includes an implantable actuator support that may be implanted in artificial passageway 219 to support actuator 340 therein. The actuator support is configured to provide the surgeon with the ability to replace implanted actuator 340 post implantation. In the exemplary embodiments described below, the actuator support is disposed in the artificial passageway between the actuator and the mastoid bone thereby preventing osseointegration of the actuator. The actuator support is configured to osseointegrate and, therefore, provides structural support for the actuator. The actuator support and the actuator are configured to enable the actuator to be removable secured to the actuator support. Thus, the actuator support may remain implanted for a substantially long time period of time during which the actuator may be replaced from time to time.

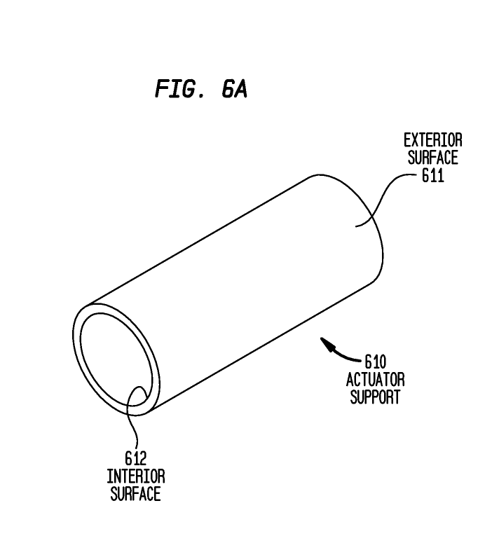

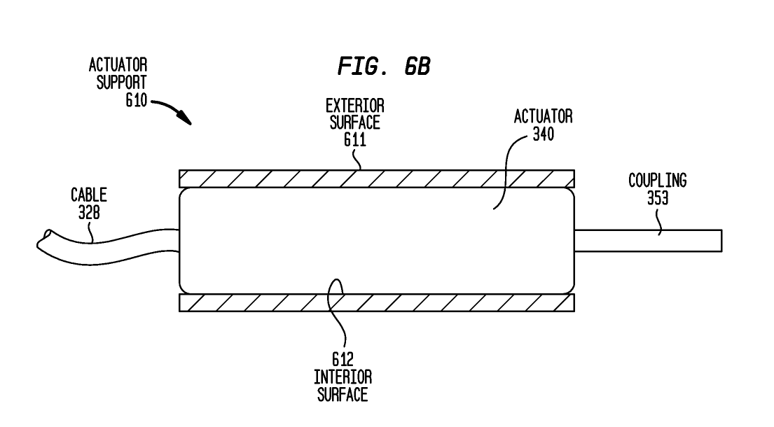

FIG. 6A is a perspective view of an exemplary embodiment of an implantable actuator support 610 in the form of a tube having an exterior surface 611 configured to osseointegrate to mastoid bone 221 and an interior surface 612 configured to receive actuator 340. FIG. 6B is a perspective view of actuator support 610 with actuator 340 disposed therein. The interior diameter of actuator support 610 is approximately the same as the outer diameter of the cylindrical body of actuator 340. Similarly, the outer diameter of actuator support 610 is approximately the same as the inner diameter of artificial passageway 219. The length of actuator support 610 substantially corresponds to the length of actuator 340, as may be seen in FIG. 6B.

As noted, actuator support 610 and actuator 340 are configured to enable the actuator to be removable secured to the actuator support. This interlock may be, in some embodiments, sufficient to prevent actuator 340 from substantially moving from the retained location in artificial passageway 219 during operation. For example, the interlock provides retention sufficient to withstand reaction forces resulting from operation of actuator 340. In an exemplary embodiment, the retention is such that the actuator 340 may be moved from the retained location upon application of a sufficient force to actuator 340 without moving actuator support 610 relative to mastoid bone 219 while maintaining or otherwise not substantially degrading osseointegration between actuator support 610 and mastoid bone 219.

In this exemplary embodiment, the interlock is provided by an interference fit between inner surface 612 of actuator support 610 and an outer surface of actuator 340. In an alternate embodiment, the interlock is implemented as threads of inner surface 612 that interface with corresponding threads on the outer surface of actuator 340. In another embodiment, O-rings or the like may be used to snuggly wrap around actuator 340 and snuggly fit inside support 610. Grooves on the actuator 340 and/or on the support 610 may be included to receive the O-ring. In other embodiments, compression of the O-ring between the actuator 340 and the support 610 provides sufficient friction to retain the components in the support 610. In another embodiment, actuator support 610 or actuator 340 include a biased extension that is adjusted against the bias to insert the actuator into the support. The extension may engage a detent on the opposing surface to interlock the actuator and support. Other embodiments include protrusions and corresponding channels on opposing surfaces of the actuator and support. An exemplary embodiment includes a spring-loaded detent that interfaces with a detent receiver of the opposing surface to hold the actuator in the support or that extends behind the actuator once the actuator has been positioned beyond the detent. An alternate embodiment may utilize O-rings to interlock the actuator in the support. Adhesive may be used to interlock the actuator in the support. Any device, system or method that will interlock actuator in the support that will permit embodiments detailed herein and/or variations thereof to be practiced may be utilized in some embodiments.

Actuator support 610 may be implanted with actuator 340 (e.g., with actuator 340 inside of implanted actuator support 610) or after implantable actuator support 610 is implanted (including inserting actuator 340 into the artificial passageway 219, followed by implantation of support 610, followed by pulling actuator 340 back into artificial passageway 219 into support 610). Implantable actuator support 610 may be implanted prior to implantation of actuator 340, followed by a period in which the support 610 is allowed to sufficiently osseointegrate to the mastoid bone, after which the actuator 340 may be attached to the support 610.

It is noted that in an exemplary embodiment, installation tool 470 may be configured to operate the particular interlock features to implant or explant actuator 340.

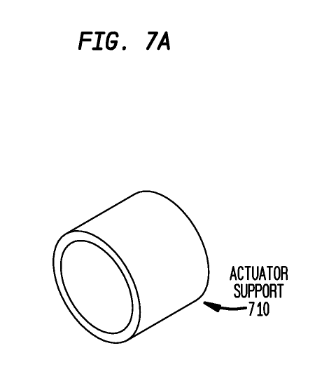

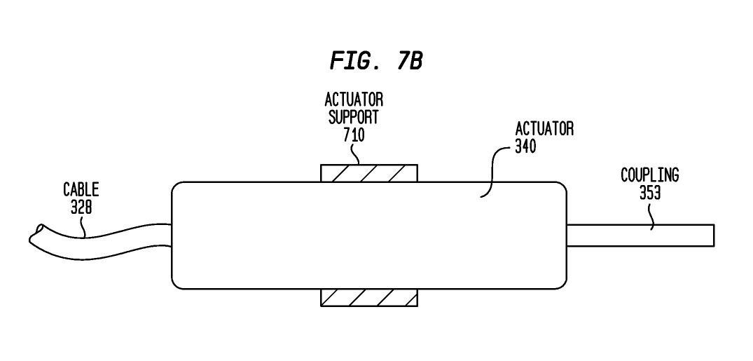

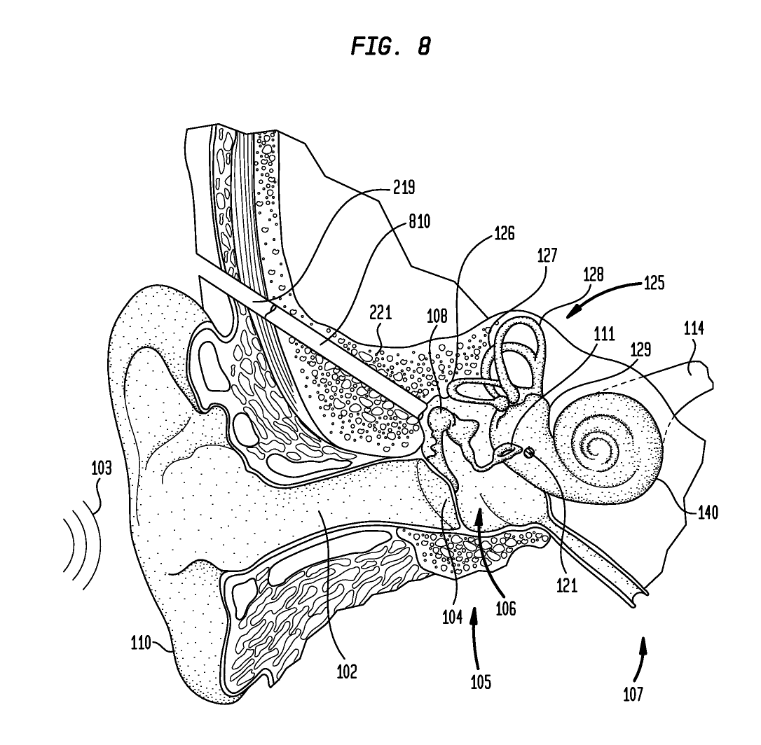

FIG. 7A depicts an alternate arrangement of an implantable actuator support 710. In an exemplary embodiment, support 710 corresponds to support 610, except that its length is shorter. Specifically, the length is a fraction of the length of the cylindrical body of actuator 340. Accordingly, when actuator 340 is removably retained within implantable actuator support 710, as may be seen in FIG. 7B, portions of the surface of the cylindrical body of actuator 340 are not covered by support 710.

FIG. 7B is representative of another embodiment in which an implantable component, such as actuator 340 has an outer surface (e.g., outer wall of the cylindrical body of actuator 340). At least a portion of the outer surface is configured to abut bone of a recipient (e.g., the bone forming the wall of artificial passageway 219 in mastoid bone 221). The outer surface that is configured to abut bone includes a first sub-portion configured to osseointegrate with a mastoid bone (e.g., the portion represented, in exaggerated form, by element 710 in FIG. 7B) and a second sub-portion (e.g., the portions of the outer surface of the cylindrical body of actuator 340 on either side of element 710 that interface with bone) configured to, relative to the first sub- portion, resist osseointegration with the mastoid bone.

In an exemplary embodiment, the first sub-portion represented by element 710 is established by surface treating actuator 340, at the first sub-portion, with a substance that enhances osseointegration. In an exemplary embodiment, the second sub-portion represented by the portions of actuator cylinder on either side of and parallel to element 710 are surface treated with a substance that reduces osseointegration relative to untreated surfaces. In an exemplary embodiment, the first sub-portion is treated with the osseointegration enhancing substance and the second sub-portion is treated with the osseointegration reducing substance.

The embodiment depicted in FIG. 7B depicts a first sub-portion 710 extending fully around a circumference of the actuator 340, although in other embodiments, the first sub-portion 710 may extend only partially around, including substantially fully around, the circumference of actuator 340.

In an exemplary embodiment, by controlling the surface area subject to osseointegration by limiting application of the substances just detailed, a retention force necessary to retain actuator 340 in artificial passageway 219 to react against forces resulting from the operation of the actuator may be obtained. This may be done while also obtaining a level osseointegration that permits the actuator 340 to be more easily removed from artificial passageway 219 than if a higher level of osseointegration were present. Such may be the case if, for example, the entire surface of the cylindrical body of actuator 340 were to be treated with the above-mentioned osseointegration substance.

In an exemplary embodiment, the first sub-portion encompasses an area having a value within a range of about ⅕th to about ⅔rds of the surface area of a first surface of actuator 340. In an exemplary embodiment, the first sub-portion encompasses an area having a value of about ⅕th, about ¼th, about ⅓rd, about ⅜ths, about ½, or about ⅔rds, or about any value in between these values in about 1/20th increments, of the surface area of a first surface of actuator 340. In an exemplary embodiment, the first surface of actuator 340 corresponds to the surface of the cylindrical body of the actuator 340 extending along the longitudinal axis. That is, it does not include the surfaces at the ends of actuator 340.

FIG. 8 depicts an alternate embodiment in which tube 810 extends substantially from (including from) a surface of mastoid bone 221 or thereabouts (i.e., the side of the mastoid bone 221 facing the outer skin of the recipient) to the inner ear cavity 423 of the recipient. In an exemplary embodiment, tube 810 corresponds to an elongated version of implantable actuator support 610 described above and is substantially longer than the length of the cylindrical body of actuator 340. In an exemplary embodiment, actuator 340 may be removably retainably received in tube 810, and cable 328 extends from actuator 340, through tube 810 to stimulator unit 320 which is located adjacent mastoid bone 221. In an exemplary embodiment, actuator 340 is implanted by driving actuator 340 through tube 810. Tube 810 may osseointegrate to the mastoid bone 221 and thus may be implanted for the post-implant lifetime of the recipient, and may also permit actuator 340 to be relatively easily explanted.

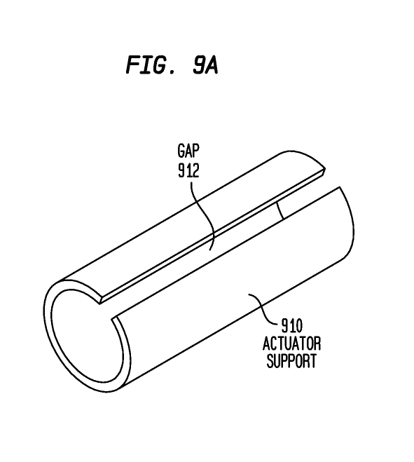

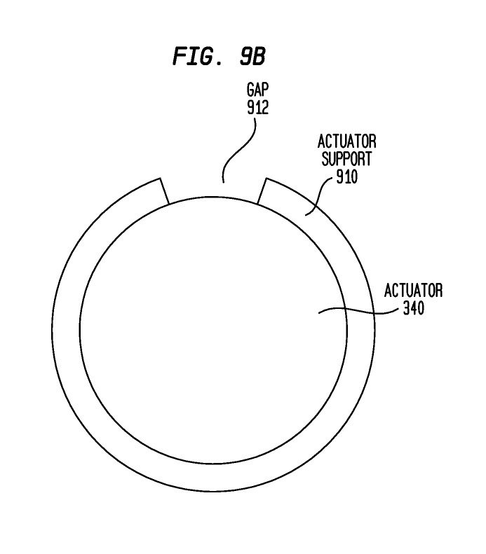

FIGS. 9A and 9B depict another exemplary embodiment of an implantable actuator support 910. Implantable actuator support 910 is an elongate body having a “C” shape cross-section lying on a plane normal to a longitudinal axis thereof, although in other embodiments, it may have a “U” shape cross section lying on that plane. Implantable actuator support 910 is configured to elastically bias legs of the “U” or “C” of the cross-section against the wall of artificial passageway 219 in a mastoid bone of the recipient, thereby removably retaining the implantable actuator support within the artificial passageway. In this regard, in a relaxed state, a width of gap 912 has a first value W1. During insertion, the with of gap 912 is reduced to a value W2 less than W1 by elastically deforming support 910, and thus the outer diameter of the support 910 is reduced to a value that permits the support 910 to be driven into artificial passageway 219. Upon placement of support 910 at the desired location in cavity 219, the gap 912 is permitted to expand to a value greater than W2 but less than W1 and thus removably retaining the implantable actuator support within the artificial passageway as a result of the spring force applied by the outer surface of support 910 to the wall of artificial passageway 219. In this expanded state, the implantable actuator support 910 is configured to removably retainably receive actuator 340 in between the legs of the “U” or “C” of the cross-section.

It is noted that in an exemplary embodiment, the gap 912 may not expand from W2. Instead, W2 represents a gap width that permits the support 910 to be driven through artificial passageway 219 but also permits actuator 340 to be retained in support 910 with a sufficient force to react against actuation forces resulting from actuation of actuator 340.

While various embodiments of the present invention have been described above, it should be understood that they have been presented by way of example only, and not limitation. It will be apparent to persons skilled in the relevant art that various changes in form and detail may be made therein without departing from the scope of the invention. Thus, the breadth and scope of the present invention should not be limited by any of the above-described exemplary embodiments, but should be defined only in accordance with the following claims and their equivalents.

What is claimed is:

1. A method of implanting a hearing prosthesis in a middle ear cavity of a recipient, comprising:
boring an artificial passageway through the temporal bone to provide access to the middle ear cavity, wherein the boring is based on a virtual model of at least a portion of a recipient's temporal bone.

2. The method of claim 1, wherein the action of boring the artificial passageway comprises:
establishing an artificial guidance system based on the virtual model; and
guiding one or more of boring direction and boring depth utilizing the artificial guidance system.

3. The method of claim 2, wherein the artificial guidance system comprises at least one of a mechanical guidance system or an electronic guidance system.

4. The method of claim 1, wherein the action of boring the artificial passageway comprises:
boring the artificial passageway utilizing a robot; and
guiding the robot during the boring based on the virtual model.

5. The method of claim 1, further comprising:
positioning a middle ear implant actuator in the passageway;
moving the actuator through the passageway to a location at which at least a portion of the actuator is disposed in the middle ear cavity;
moving a stapes prosthesis from an outer ear of the recipient into the middle ear cavity; and
connecting the stapes prosthesis, while in the middle ear cavity, to a coupling element of the middle ear implant actuator.

6. The method of claim 1, wherein the action of moving the positioned actuator through at least a portion of the artificial passageway comprises:
utilizing an insertion device that cradles a receiver unit and a stimulator unit of an implantable component of a middle ear implant to move the actuator through the artificial passageway while the stimulator unit is connected to the actuator via an implantable cable.

7. A hearing prosthesis, comprising:
an implantable actuator support having a first surface configured to osseointegrate to a mastoid bone of a recipient and a second surface configured to removably receive a middle ear implant actuator.

8. The hearing prosthesis of claim 7, wherein:
the implantable actuator support is a tube;
an outer surface of the tube corresponds to the first surface; and
an inner surface of the tube corresponds to the second surface.

9. The hearing prosthesis of claim 7, wherein:
the second surface is configured to removably retainably receive the middle ear implant actuator.

10. The hearing prosthesis of claim 9, wherein:
the second surface is configured to removably retainably receive the middle ear implant actuator via an interference fit between the second surface and an outer surface of the middle ear implant actuator.

11. The hearing prosthesis of claim 7, wherein:
the second surface is configured to removably retainably receive the middle ear implant actuator via threads that interface with corresponding threads on an outer surface of the middle ear implant actuator.

12. The hearing prosthesis of claim 7, wherein:
the implantable actuator support includes a spring apparatus configured to removably retainably receive the middle ear implant actuator.

13. The hearing prosthesis of claim 7, wherein:
the implantable actuator support is a tube configured to extend substantially from a first surface of a mastoid bone proximate outer skin of the recipient to a middle ear cavity of the recipient.

14. The hearing prosthesis of claim 7, wherein:
the implantable actuator support is an elongate body having a “U” shape or a “C” shape cross-section lying on a plane normal to a longitudinal axis thereof;
the implantable actuator support is configured to elastically bias legs of the cross-section against a wall of an artificial passageway in a mastoid bone of the recipient, thereby removably retaining the implantable actuator support within the artificial passageway; and
the implantable actuator support is configured to removably retainably receive a middle ear implant actuator in between the legs of the cross-section.

15. A hearing prosthesis, comprising:
an implantable component having an outer surface, wherein at least a portion of the outer surface is configured to abut bone of a recipient, the outer surface including a first sub-portion configured to osseointegrate and a second sub-portion configured to, relative to the first sub- portion, resist osseointegration.

16. The hearing prosthesis of claim 15, wherein:
the first sub-portion extends fully or substantially fully around a circumference of the implantable component.

17. The hearing prosthesis of claim 15, wherein:
the implantable component includes a portion having a substantially cylindrical shape having a longitudinal axis; and
the first surface corresponds to the surface of the cylindrical shape extending along the longitudinal axis.

18. The hearing prosthesis of claim 17, wherein:
the first sub-portion encompasses an area having a value within a range of about ⅕th to about ⅔rds of the surface area of the first surface.

19. The hearing prosthesis of claim 15, wherein the implantable component includes at least a first sub-component and a second sub-component, the first sub-component being removably retained by the second sub-component, the first sub-portion being part of the first sub-component and the second sub-portion being part of the second sub-component.

20. The hearing prosthesis of claim 19, wherein:
the second sub-component corresponds to a middle ear actuator having a cylindrical body; and
the first sub-component corresponds to one of an annular body or a substantially annular body configured to extend about the cylindrical body of the middle ear actuator.
